(19) United States
(12) Patent Application Publication
(10) Pub. No.: US 2019/0069773 A1
(43) Pub. Date: Mar. 7, 2019

(54) SYSTEM AND METHOD FOR DETECTING EARLY VISION EYE PROBLEMS

(71) Applicant: Thomas R. Cataldo, Anaheim, CA (US)

(72) Inventor: Thomas R. Cataldo, Anaheim, CA (US)

(21) Appl. No.: 15/698,398

(22) Filed: Sep. 7, 2017

(51) Int. Cl. A61B 3/032

(52) U.S. Cl. CPC A61B 3/032; A61B 2503/12; A61B 2503/06; A61B 2503/04

(57) ABSTRACT

A system or method for detection of vision problems. The system or method may include one or more of an eye chart, eye patch, reply form, instruction sheet, and reward for returning the reply form. One or more of the eye chart, eye patch, and/or reply form may be packaged together within a single envelope for delivery to a potential user. In order to be effective with individuals that are not well versed in language or numbers, such as infants, children, adults with certain learning disabilities, or others (e.g., adults not fluent in a particular language, such as English), the eye chart may include only graphical elements for visual identification in the vision test by the individual. One or more different eye charts, eye patches or rewards may be provided to a particular user based upon determinations made about the particular user (e.g., age, sex, interests, etc.).

### BACKGROUND

### 1. Field of the Invention

The present invention relates to a system and method for detecting vision problems. More particularly, the present invention relates to a system and method for early detection of vision problems in individuals, particularly children, who may not be versed in language, letters or numbers.

### 2. Description of the Related Art

Vision or sight disorders are among the most significant conditions that impact and handicap quality of life for individuals, particularly in children or infants. Untreated vision problems often lead to a variety of negative life experiences, including physical discomfort, permanent vision loss, and limitations in academic performance. Indeed, a high percentage of learning in children and adults is achieved through their visual processing of information. As such, those with vision or sight disorders are more likely to struggle with educational, and other, tasks. Reading is particularly impacted for such children and adults, through vision problems often lead to a host of issues, resulting in significantly more mistakes than those not affected by vision problems, copying of notes, misaligning of numbers in columns, and struggling with filling out test forms to record answers to standardized exams. Studies have underscored the importance of vision to academic achievement, particularly in elementary school students, finding that visual acuity is a greater predictor of performance on specific standardized tests, more so than race or socioeconomic status.

Similarly, a constant strain on the eyes can lead to tension in the face, neck, and shoulders, which can further impact educational capabilities. Students with vision impairments often develop headaches, nausea, dizziness, and other discomforts that can decrease a child's ability to concentrate in school. Moreover, such impairments also contribute to safety concerns, such as falling, tripping and running into objects. Consequently, children may act out or misbehave because of their struggles to see and focus on their school tasks. In part due to these problems, it is recommended that children receive a comprehensive eye exam by a vision professional early and be regularly tested thereafter. However, various barriers keep this from occurring with only roughly one third of young children across the country actually receive eye exams before starting school.

In addition, bullying in school, and in other social circles, place additional stress upon children, and is often exacerbated for children with vision impairments. A variety of negative effects result from bullying, including lower self-esteem, feelings of fear, school phobia and absenteeism, nightmares and sleeplessness, depression and anxiety, and even suicidal thoughts. Unfortunately, conventional vision tests are often not feasible or not performed for children at an early enough stage to correct serious problems. In some cases, particularly for low-income families, early detection of vision problems may not be sought for children at all. In such circumstances, irreparable damage may occur, leading to lifelong vision impairments that could have been corrected if detected at an early stage.

In light of one or more of the above issues, it would be desirable to provide or implement a system or method for early detection of vision impairments in children. Such a system or method would ideally allow for children with limited language capabilities to indicate whether vision impairment is present or not. In addition, such a system or method would be convenient and inexpensive to conduct.

### SUMMARY

The present invention is related to a system or method that helps detect vision problems, particularly early detection, such as in infants or children. In one embodiment, a system for aiding in early detection of vision problems may include an eye chart including only pictorial elements for identification by a user, an eye patch configured to be worn by the user, a reply form configured to be modified after the user has used the eye chart, and a reward configured to be provided to the user based upon the reply form being modified after the user has used the eye chart, wherein the eye chart, the eye patch, and the reply form are packaged within a single envelope for provision to an address associated with the user.

In another embodiment, a system for aiding in early detection of vision problems may include a first eye chart including first graphical elements for visual identification, the first graphical elements configured for a first user having a first characteristic, a second eye chart including elements for visual identification, the second elements configured for a second user having a second characteristic, and an eye patch configured to be worn by the first user.

In still another embodiment, a method for aiding in early detection of vision problems may include providing an eye chart to a user, providing an eye patch to the user, providing a reply form to the user, receiving the reply form back from the user, and providing a reward to the user based upon the reply form being received from the user, wherein the eye chart, the eye patch, and the reply form are provided via a single envelope for provision to an address associated with the user.

### BRIEF DESCRIPTION OF THE DRAWINGS

The features, objects, and advantages of the present invention will become more apparent from the detailed description set forth below when taken in conjunction with the drawings, wherein:

FIG. 1 shows an eye chart for aiding in early detection of vision problems, according to an embodiment of the present invention;

FIG. 2 shows an eye chart for aiding in early detection of vision problems, according to an embodiment of the present invention;

FIG. 3 shows a feedback or reply form associated with a system for aiding in early detection of vision problems, according to an embodiment of the present invention;

FIG. 4 shows a flowchart for a method for aiding in early detection of vision problems, according to an embodiment of the present invention; and

FIG. 5 shows an eye patch for aiding in early detection of vision problems, according to an embodiment of the present invention.

### DETAILED DESCRIPTION

The detailed description of exemplary embodiments herein makes reference to the accompanying drawings and pictures, which show the exemplary embodiment by way of illustration and its best mode. While these exemplary embodiments are described in sufficient detail to enable those skilled in the art to practice the invention, it should be understood that other embodiments may be realized and that logical and mechanical changes may be made without departing from the spirit and scope of the invention. Thus, the detailed description herein is presented for purposes of illustration only and not of limitation. For example, the steps recited in any of the method or process descriptions may be executed in any order and are not limited to the order presented. Moreover, any of the functions or steps may be outsourced to or performed by one or more third parties. Furthermore, any reference to singular includes plural embodiments, and any reference to more than one component may include a singular embodiment.

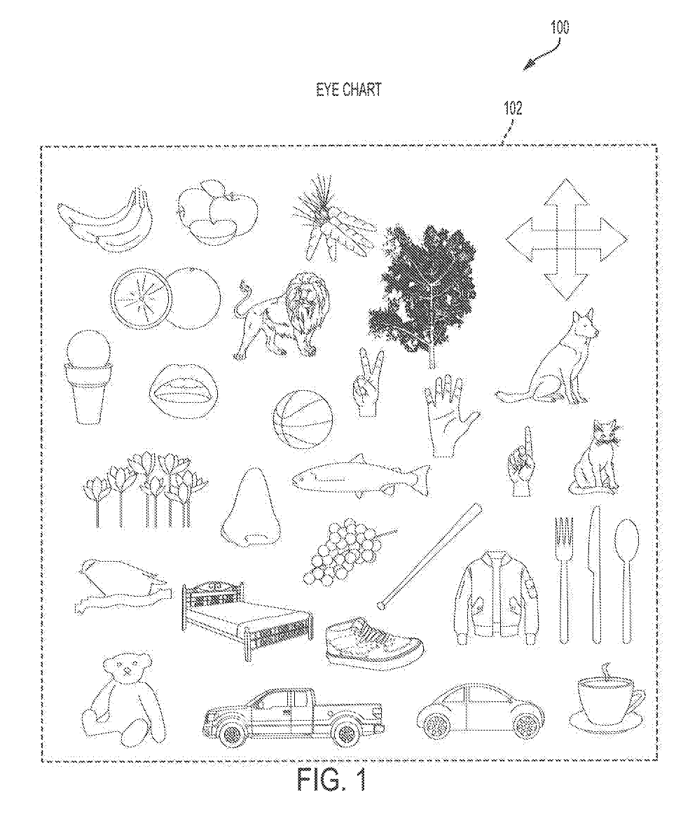

FIG. 1 illustrates an eye chart 100 for aiding in early detection of vision problems. The eye chart 100 may be used as part of a system or method configured to allow for children, or other individuals, to test for vision-related problems at home. For example, as discussed in greater detail herein, the eye chart 100 may be provided (e.g., mailed), with or without additional materials, so that initial testing for vision problems may be performed at a home or residence of the user, rather than initial testing requiring appointment or meeting at a clinic, doctor office, or other health-based facility.

The eye chart 100 includes a variety of graphical (e.g., pictorial, icons, etc.) elements 102. In one embodiment, the graphical elements 102 may be images without any text, numbers, and/or other indicia that would not be easily understandable by a certain demographic of user. For example, as previously discussed, diagnosing of vision problems in infants or children, although medically critical for early detection of vision-related problems, can be difficult or impossible in many circumstances since infants or children may be unable to comprehend the language, letters, or numbers of traditional eye charts or vision tests. Accordingly, the eye chart 100 may include only graphical or image-based elements 102 in order to better support provision of vision testing to early adolescents and allow for testing even to those individuals who would not normally understand. In another example, adults may similarly benefit from the eye chart 100 with only graphical elements 102, especially if a particular adult is not well versed in a particular language (e.g., English) for which a traditional eye chart or vision test has been created. Alternative embodiments may utilize elements that include a mix of graphical images with text and/or numerical elements.

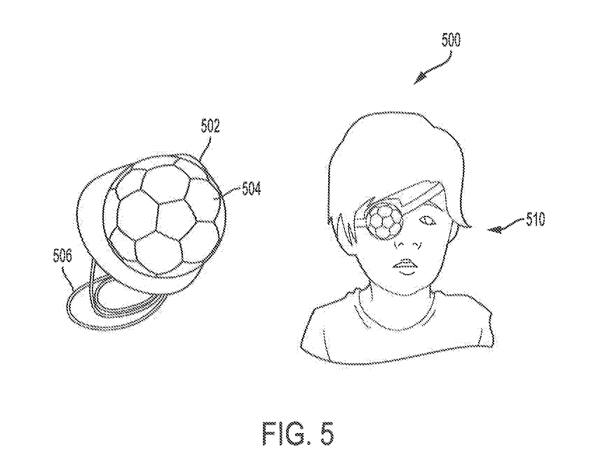

In operation, the eye chart 100 would be displayed to the user. The user would cover one eye (e.g., with their hand, an eye patch (e.g., as discussed in greater detail for FIG. 5), or other component) and asked to look at and/or identify a particular element 102 on the eye chart 100. The accuracy of eye position towards the particular element 102, accuracy of other identification (e.g., pointing to the particular element 102 or other hand motions, verbal indication, etc.), time taken to look or and/or identify the particular element 102, etc. are noted. Subsequently, the user covers their other eye (e.g., with their hand, an eye patch (e.g., as discussed in greater detail for FIG. 5), or other component) and again asked to look at and/or identify a particular element 102 (e.g., the same elements previously requested and/or different elements) on the eye chart 100. The same accuracy of eye position towards the particular element 102, accuracy of other identification (e.g., pointing to the particular element 102 or other hand motions, verbal indication, etc.), time taken to look or and/or identify the particular element 102, etc. are noted again for the second eye. Differences or discrepancies in the above are noted (e.g., recorded in writing). If differences exist, or if there is no response in identifying a particular element 102 when there should be (depending upon the characteristics of the user, such as age), these notes may be taken to the user's physician/pediatrician for further analysis (e.g., referral to an ophthalmologist).

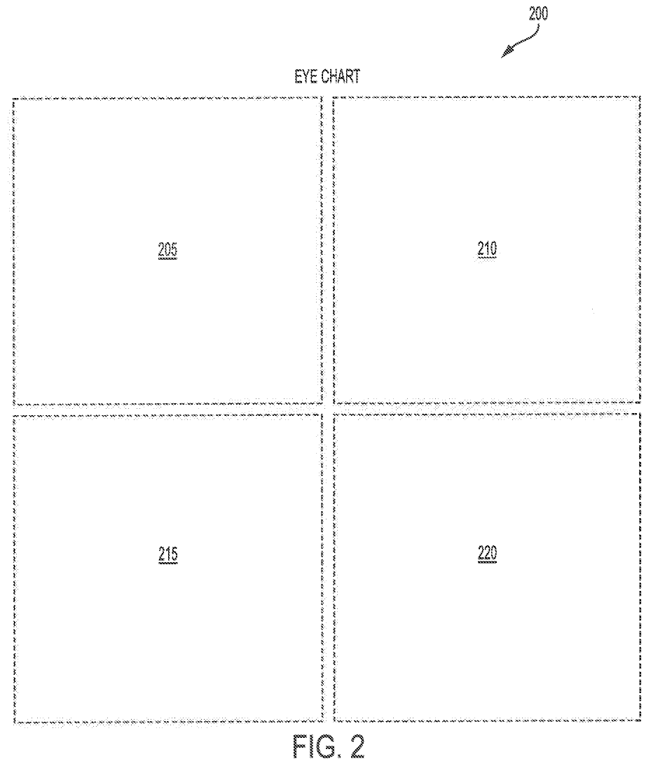

FIG. 2 illustrates an eye chart 200 for aiding in early detection of vision problems. The eye chart 200 may include certain features and/or be used in similar manners to the eye chart 100, previously discussed for FIG. 1. The eye chart 200, however, includes a plurality of sections (205, 210, 215, 220) that may contain different elements (e.g., textual, graphical, etc.). For example, in one embodiment, a first section 205 may include elements that are configured to be interacted with by a user having first characteristics (e.g., a user having a particular age or age range, having a particular sex, living in or associating with a particular geographical location, or any of a variety of other characteristics or proclivities or interests). A second section 210 may include elements (e.g., different elements and/or some of the same elements) that are configured to be interacted with by a user having second characteristics. A third section 215 may include elements (e.g., different elements and/or some of the same elements) that are configured to be interacted with by a user having third characteristics. A fourth section 220 may include elements (e.g., different elements and/or some of the same elements) that are configured to be interacted with by a user having fourth characteristics.

Thus, the same eye chart 200 may be configured to be used by a variety of users having different characteristics, proclivities, and/or interests. For example, rather than having to manufacture and/or send a different eye chart to a particular user based upon determining one or more certain characteristics of that particular user (e.g., whether the user is within a certain age range, whether the user speaks English, whether the user has an interest in sports, etc.) the same eye chart 200 may be used by all such users. Accordingly, for example, if the first section 205 included elements configured for use by a child under the age of three (e.g., contains elements corresponding to pictures of animals, human body parts, etc.) and if the second section 210 included elements for use by a child over the age of three (e.g., contains elements corresponding to pictures of other objects that might not be easily recognizable to a child under the age of three), the same eye chart 200 may still be provided to either such user, each user's vision test (for example, as previously discussed for FIG. 1) focusing only on those particular sections (205, 210, 215, 220) that correspond to the user taking the test.

As previously discussed, a system or method for aiding in detection (e.g., early detection) of vision problems may be configured to be performed at a user's home or place of residence, rather than requiring the user to take an initial test at a medical professional facility or place of business. For example, such a home administration system or method may include one or more eye charts, eye patches, etc., as discussed in greater detail throughout. The entity in charge or managing the distribution, creation, manufacture, or having other involvement with the home administration system or method may seek feedback from users to whom the system has been provided to.

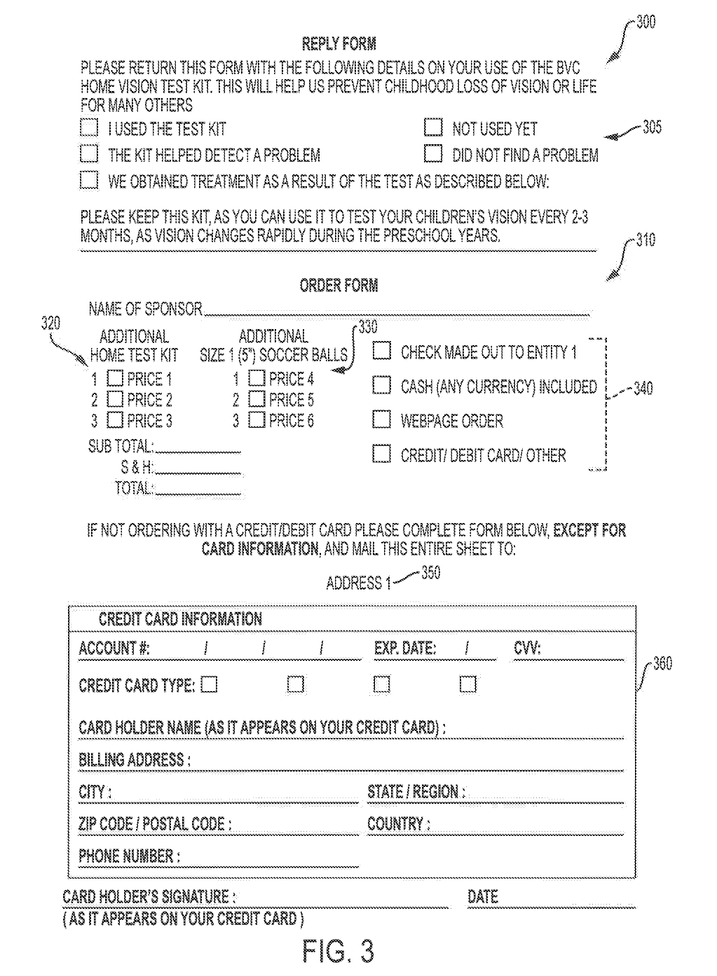

FIG. 3 shows a feedback or reply form 300 associated with such a home administration system or method. Certain features of the system or method may be the same or similar to those previously discussed. The form 300 includes address information 350 that instructs the user on where and/or how to return the form to the appropriate entity. The form 300 may include a first section 305 for a user, or someone on the user's behalf, to fill out regarding how the system or method was performed. For example, options to select may include “I used the test kit,” “The Kit helped detect a problem,” We obtained treatment as a result of the test, as described below” and include a section for additional information regarding the obtained treatment, “Not used yet,” “Did not find a problem,” etc. Thus, even if the system or method does not end up being used by a user after its provision to the user, the form 300 may still be filled-out and returned to the appropriate entity seeking feedback. In order to encourage fill-out and return of the form 300, a sponsor (e.g., a pre-contracted or predetermined business, company, corporation, group, individual, or other entity) may agree to provide a special “reward” to a user for sending back the form 300. The reward may be in the form of an object (e.g., a toy or other product) or may be in the form of a coupon or discount for a product or service offered by the sponsor or other entity. In certain embodiments, multiple rewards and/or multiple sponsors may participate and users may be able to choose among the rewards and/or sponsors in return for filling out and returning the form 300.

The form 300 may also include a second section 310 relating to the ordering of additional components associated with the system or method for detection of vision problems. For example, although one test kit (e.g., eye chart, eye patch, instructions, etc.) may have already been provided to a user, additional (e.g., same and/or different) test kits may be desired. In one such instance, a first test kit may have been provided (e.g., mailed, handed-out, etc.) for use by a first particular user (e.g., a male child under the age of three). There may be additional users, known to the first particular user, for which a test kit would also be desired (e.g., family members, friends, etc.). Thus, the second section 310 of the form 300 may include a test kit ordering section 320 for ordering one or more additional test kits. These test kits may be the same (e.g., eye charts, eye patches, etc. that are the same as the one already received) or, in certain embodiments, may be different (e.g., eye charts for a different particular user based on such user's characteristics, such as age, sex, interests, etc.). A corresponding price may correspond to the ordering of the one or more additional test kits in the section 320.

In certain embodiments, a sponsor (e.g., a pre-contracted or predetermined business, company, corporation, group, individual, or other entity) may agree to provide a special “reward” to a user for sending back the form 300, as previously discussed. It may be desired for additional of these rewards (or other rewards provided by a sponsor) to be purchased by the user (e.g., if a household has multiple users of the Test Kit and each user would desire one of the rewards). Thus, the second section 310 of the form 300 may include a reward ordering section 330 for ordering one or more additional rewards (e.g., soccer balls as exemplary illustrated in FIG. 3). Payment for the additional reward(s) (e.g., via section 330) and/or the additional test kit(s) (e.g., via section 320) may be configured via a payment option section 340 that may include various options for providing the corresponding payment (e.g., check, cash, webpage order, credit/debit card/other). In the case of a credit and/or debit card payment option being selected, a card information section 360 may be provided that allows the user to fill in the corresponding card information in order to effect payment using the user's card of choice.

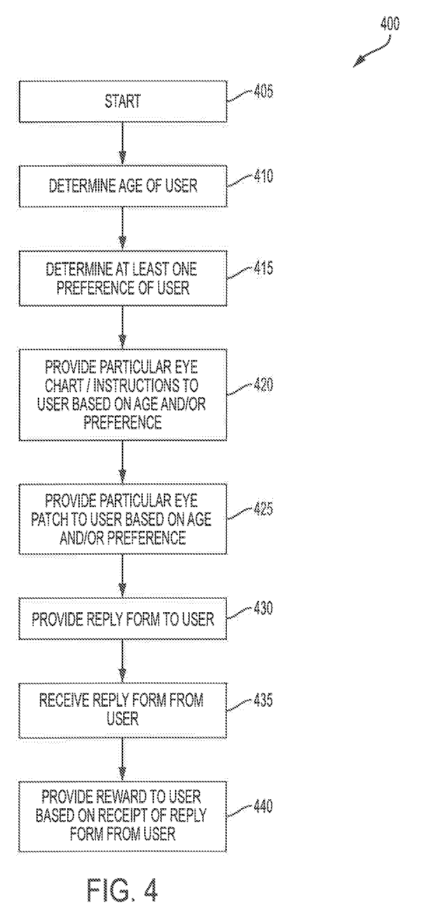

FIG. 4 illustrates a flowchart for a method for aiding in early detection of vision problems. Certain features of the method and/or an associated system incorporating the method may be the same or similar to those previously discussed. The method of flowchart 400 starts at step 405 where the initial reach-out and/or engagement of a potential user is initiated. This start may occur by any of a variety of methods (e.g., telephone call, in-person discussion, communication by mail, communication by email, communication via real-time chat, communication via text, loading of a software application, such as an application for a smart phone or tablet or computer, etc.). At step 410 the age of the user who is to be using the vision test is determined. The age may be a discrete number and/or may be defined as within a predetermined range (e.g., the user is “under the age of three,” the user is “between the ages of three and five,” etc.). At step 415, at least one preference of the user who is to be using the vision test is determined (e.g., the user “enjoys sports,” the user “enjoys animals,” etc.

At step 420, a particular eye chart (e.g., eye chart 100 and/or 200, as previously discussed) is provided for the user's use based upon the age of the user determined at step 410 and/or the preference(s) of the user determined at step 415. For example, if at step 410 it is determined that the user is under the age of three and it is determined at step 415 that the user has a preference towards animals, a particular eye chart that includes only pictures or images (e.g., rather than text due to the young age of the user) of animals (e.g., rather than other images that may not yet be known, recognizable, or preferred by the user due to the user's preference) may be provided. Provision of the eye chart may be by way of physical mail to an address specified for the user, by way of electronic transmission (e.g., email, messaging, software application, etc.). For example, in one embodiment, a mobile application (e.g., for a smartphone, tablet, or other electronic device) may display the appropriate eye chart for use by the user.

At step 425, a particular eye patch (e.g., an eye patch as further discussed in FIG. 5) is provided for the user's use based upon the age of the user determined at step 410 and/or the preference(s) of the user determined at step 415. For example, if at step 410 it is determined that the user is under the age of three and it is determined at step 415 that the user has a preference towards animals, a particular eye patch that includes a picture, image, logo, other feature, etc. associated with an animal and/or promoting a particular sponsor (e.g., a sponsor associated with younger children) may be provided. Provision of the eye patch may be by way of physical mail to an address specified for the user or by way of electronic transmission (e.g., email, messaging, software application, etc.) to the user instructing the user to pickup the eye patch at a designated location (e.g., a particular store or establishment). In one embodiment, the eye chart of step 420 and the eye patch of step 425 may be provided together to the user (e.g., packaged within the same physical envelope).

At step 430, a reply or feedback form is provided to the user. The form may allow the user or someone on behalf of the user to provide feedback or other information based upon their usage of the eye chart and/or eye patch. Provision of the form may be by way of physical mail to an address specified for the user or by way of electronic transmission (e.g., email, messaging, software application, etc.). In one embodiment, the form and the eye chart of step 420 and the eye patch of step 425 may all be provided together to the user (e.g., packaged within the same physical envelope). At step 435, the reply or feedback form provided to the user at step 430 may be received back from the user, with certain information filled in or otherwise indicated on the form, for example, based on the user's experience in using one or more of the eye chart or eye patch. At step 440, a reward (e.g., toy, product, coupon, service, or other benefit) is provided to the user person(s) associated with the user based on the receipt of the form via step 435. As previously discussed, this reward may be provided by a sponsor and/or partner that has been pre-contracted or predetermined for provision of the reward.

FIG. 5 illustrates an eye patch 500 for aiding in early detection of vision problems. Certain features of the eye patch 500 and/or the system or method associated with the early detection of vision problems that utilizes the eye patch 500 may be the same or similar to those previously discussed. The eye patch 500 may include a portion 502 shaped to fit over an eye of a user and a strap 506, attached to the portion 502, and configured to secure the portion 502 to the user (e.g., around a portion of the user's head) such that the user may wear 510 the eye patch 500. Graphics or other visual elements may be disposed upon the portion 502 and/or the strap 506. Any of a variety of such visual elements (logos, sponsorship information, pictures, etc.) may be provided upon the portion 502 and/or the strap 506 in various embodiments. For example, the graphics may be customized based upon or for one or more user's particular characteristics (e.g., age, sex, interests, etc.). In such an embodiment, the same or similar as previously discussed for provision of a particular eye chart provided to a particular user based upon user characteristics, a particular eye patch for such particular user may also be provided (e.g., a male child under the age of three may receive an eye patch with different graphical elements than a female child over the age of three).

The various features and/or components described above for the various figures (e.g., eye chart, eye patch, reply form, etc.) may be provided together in one packet or kit (e.g., mailed envelope) in certain embodiments, together with instructions and/or any other desired materials. In certain embodiments, rather than a physical envelope mailing physical materials to a user, a software application (e.g., upon a smart phone, tablet, computer, etc.) may contain the eye chart, instructions, reply form, etc. such that a user can perform a vision test using only their electronic device.

For example, the software application may be configured to run upon a handheld or mobile device and/or may be configured to transmit data to a remote server (e.g., a server operated by an owner or manager of the software application, to a doctor or health care facility, etc.). In one embodiment, a user that uses the software application (e.g., which may involve or display an eye chart having some or all of the features previously discussed), may input data regarding their interaction with the software application (e.g., information concerning whether a left or right eye of an individual using the eye chart exhibited any discrepancies, such as taking a longer period of time to identify an item on the eye chart or not identifying an item on the eye chart at all with either the left or the right eye). Thus, in certain embodiments, rather than relying on a user to manually make an appointment with a health care provider and describe the potential issues with such provider, the software application may be configured to automatically transmit data concerning the eye chart results to a participating healthcare provider and/or to another remote server or location that may then forward, transmit, or otherwise take action to setup further health care investigation for the user.

The previous description of the disclosed examples is provided to enable any person of ordinary skill in the art to make or use the disclosed methods and apparatus. Various modifications to these examples will be readily apparent to those skilled in the art, and the principles defined herein may be applied to other examples without departing from the spirit or scope of the disclosed method and apparatus. The described embodiments are to be considered in all respects only as illustrative and not restrictive and the scope of the invention is, therefore, indicated by the appended claims rather than by the foregoing description. All changes which come within the meaning and range of equivalency of the claims are to be embraced within their scope. Skilled artisans may implement the described functionality in varying ways for each particular application, but such implementation decisions should not be interpreted as causing a departure from the scope of the disclosed apparatus and methods. The steps of the method or algorithm may also be performed in an alternate order from those provided in the examples.

What is claimed is:

1. A system for aiding in early detection of vision problems comprising:
an eye chart including only pictorial elements for identification by a user;
an eye patch configured to be worn by the user;
a reply form configured to be modified after the user has used the eye chart; and
a reward configured to be provided to the user based upon the reply form being modified after the user has used the eye chart,
wherein the eye chart, the eye patch, and the reply form are packaged within a single envelope for provision to an address associated with the user.

2. The system of claim 1 wherein the pictorial elements of the eye chart are configured to a user within a particular age range.

3. The system of claim 1 wherein the pictorial elements of the eye chart are configured to a user having a particular interest.

4. The system of claim 1 wherein the pictorial elements of the eye chart are configured to a user of a particular sex.

5. The system of claim 1 wherein the eye patch is configured to a user within a particular age range.

6. The system of claim 1 wherein the eye patch is configured to a user having a particular interest.

7. The system of claim 1 wherein the eye patch is configured to a user of a particular sex.

8. A system for aiding in early detection of vision problems comprising:
a first eye chart including first graphical elements for visual identification, the first graphical elements configured for a first user having a first characteristic;
a second eye chart including elements for visual identification, the second elements configured for a second user having a second characteristic; and
an eye patch configured to be worn by the first user.

9. The system of claim 8 wherein the eye patch and either the first eye chart or the second eye chart are packaged within a single envelope for physical mailing to an address.

10. The system of claim 8 further comprising a reply form configured to be modified after usage of the first eye chart or the second eye chart.

11. The system of claim 10 further comprising a reward configured to be provided to the user if the user returns the reply form.

12. The system of claim 8 wherein the first characteristic and the second characteristic correspond to an age of the first user or the second user.

13. The system of claim 8 wherein the first characteristic and the second characteristic correspond to a sex of the first user or the second user.

14. The system of claim 8 wherein the first eye chart contains only graphical elements for visual identification without any textual or numerical elements for visual identification.

15. A method for aiding in early detection of vision problems comprising:
providing an eye chart to a user;
providing an eye patch to the user;
providing a reply form to the user;
receiving the reply form back from the user; and
providing a reward to the user based upon the reply form being received from the user,
wherein the eye chart, the eye patch, and the reply form are provided via a single envelope for provision to an address associated with the user.

16. The method of claim 15 wherein the eye chart only includes pictorial elements for use by the user in performing a vision test.

17. The method of claim 15 further comprising:
determining a characteristic of the user;
determining that the eye chart is to be provided to the user based upon the determined characteristic of the user.

18. The method of claim 17 wherein the characteristic is one of: an age of the user, a sex of the user, or a preference of the user.

19. The method of claim 15 further comprising:
determining a characteristic of the user; and
determining that the eye patch is to be provided to the user based upon the determined characteristic of the user.

20. The method of claim 19 wherein the characteristic is one of: an age of the user, a sex of the user, or a preference of the user.
